(19) United States
(12) Patent Application Publication
(10) Pub. No.: US 2025/0170170 A1
(43) Pub. Date: May 29, 2025

(54) BOWEL-CLEANSING COMPOSITION

(71) Applicant: DAEWOONG THERAPEUTICS INC., Hwaseong-si (KR)

(72) Inventors: Sang Han PARK, Seoul (KR); Min Hyung KANG, Yongin-si (KR); Hyun Ju CHOI, Seongnam-si (KR); Seung Jun KANG, Hwaseong-si (KR); Sun Yong YANG, Suwon-si (KR)

(73) Assignee: DAEWOONG THERAPEUTICS INC., Hwaseong-si (KR)

(21) Appl. No.: 18/521,756

(22) Filed: Nov. 28, 2023

(51) Int. Cl. A61K 33/06; A61K 31/4402; A61K 31/4525; A61K 31/795; A61K 33/04; A61P 1/10

(52) U.S. Cl. CPC A61K 33/06; A61K 31/4402; A61K 31/4525; A61K 31/795; A61K 33/04; A61P 1/10

(57) ABSTRACT

Provided is a bowel-cleansing composition comprising anhydrous magnesium sulfate, potassium sulfate, and anhydrous sodium sulfate, in combination with prucalopride or sodium picosulfate.

### TECHNICAL FIELD

The present invention relates to a bowel-cleansing composition comprising anhydrous magnesium sulfate, potassium sulfate, and anhydrous sodium sulfate, in combination with prucalopride or sodium picosulfate.

### BACKGROUND ART

For an accurate colonoscopy, a bowel-cleansing process that cleanses the intestines in advance is essential. In spite of fasting for several days, stools still remain in the intestines; and thus the intestines including the colons should be cleansed through an artificial method before colonoscopy. Bowel-cleansing preparations (or bowel-cleansing formulations) are an essential medicament that allows for accurate colonoscopy by thoroughly washing away intestinal residues such as feces in advance.

The active ingredients of bowel-cleansing preparations are divided into an osmotic laxative and a stimulant laxative depending on their mechanism of action. Osmotic laxatives include polyethylene glycol (PEG), sulfates, magnesium citrate, and so on. For example, an isotonic laxative preparation comprises polyethylene glycol (PEG) as an active ingredient, while a hypertonic laxative preparation comprises sulfates as an active ingredient. Representative stimulant laxatives include sodium picosulfate, which is conventionally used in combination with osmotic laxatives such as magnesium citrate.

Isotonic laxative preparations comprising PEG as an active ingredient can maintain osmolality in the intestinal tract without absorption into the body or irritating secretions in the colon lumen and minimize the movement of body fluids and electrolytes to avoid significant physiological changes. However, the isotonic laxative preparations have some drawbacks, such as large amounts of dose (e.g., 2 to 4 liters or more), burdens due to unpleasant taste, and so on.

Hypertonic laxative preparations comprise the sulfates such as sodium sulfate, magnesium sulfate, and potassium sulfate as active ingredients. Although hypertonic laxative preparations are easier to take than the PEG-based isotonic preparations, they have the disadvantage of causing fluid and electrolyte imbalance. Thus, the hypertonic laxative preparations are not recommended for the patients having congestive heart diseases, ascites, or severe renal failures. In addition, a conventional hypertonic laxative preparation (e.g., Orafang® tablet) contains large amounts of the sulfates in a unit dose. For example, the unit dose of Orafang® tablet includes 15.75 g of anhydrous sodium sulfate, 2.8 g of potassium sulfate, and 1.4 g of anhydrous magnesium sulfate. Therefore, conventional hypertonic laxative preparations have additional drawbacks in the facts that the size of tablets is too large and that the patients should take many tablets (the first 14 tablets and the second 14 tablets) along with 1 liter of water each time, which leads to patients' low compliance.

Combination preparations comprise sodium picosulfate as a stimulant laxative and magnesium citrate as an osmotic laxative. For example, Chemlight® Powder, one of the combination preparations, includes 10 mg of sodium picosulfate, 12.0 g of citric acid, and 3.5 g of magnesium oxide as active ingredients in a unit dose. However, the combination preparations have some side effects, such as mild digestive symptoms (e.g., abdominal cramps, pain, nausea, vomiting, etc.) and hypermagnesemia due to hyperosmolarity and high magnesium contents. Especially, since the combination preparations should be dissolved in water before the administration, the patients should intake large amount of water (250 mL per hour).

Therefore, conventional bowel-cleansing preparations require taking a large amount of unit dose and causes nausea and pains to patients. Due the difficulties, many people reject receiving colonoscopy; stop taking a bowel-cleansing preparation in an insufficient bowel-cleansing state; or expel the ingested bowel-cleansing preparation due to vomiting. Accordingly, there is a need to develop a bowel-cleansing preparation (or formulation) capable of improving patients' compliance while maintaining bowel-cleansing effects.

### DISCLOSURE

### Technical Problem

It is an object of the present invention to provide a novel bowel-cleansing composition capable of improving patients' compliance while maintaining and/or improving bowel-cleansing effects.

### Technical Solution

According to an aspect of the present invention, there is provided a bowel-cleansing composition comprising anhydrous magnesium sulfate, potassium sulfate, and anhydrous sodium sulfate, in combination with prucalopride or sodium picosulfate.

### Advantageous Effects

The composition of the present invention can reduce the contents of sodium sulfate, magnesium sulfate, and potassium sulfate, thereby reducing the side effects that may be caused therefrom (e.g., body fluid and electrolyte imbalance). The reduction of the contents thereof can lead to reducing the actually administered amount of tablet(s), thereby being capable of improving patients' compliance. In addition, the composition of the present invention exhibits similar or improved bowel-cleansing effects compared to conventional formulations, along with reducing the occurrence of side effects and improving the patients' compliance.

### DESCRIPTION OF DRAWINGS

FIG. 1 shows the results obtained by evaluating the colon cleansing scores of the bowel-cleansing compositions of Examples and Comparative Examples.

FIG. 2 shows the results obtained by evaluating the bowel-cleansing levels in the colons isolated after treating with the bowel-cleansing compositions of Examples and Comparative Examples.

### BEST MODE FOR CARRYING OUT THE INVENTION

The present invention provides a bowel-cleansing composition comprising anhydrous magnesium sulfate, potassium sulfate, and anhydrous sodium sulfate, in combination with prucalopride or sodium picosulfate.

The composition of the present invention can reduce the contents of sodium sulfate, magnesium sulfate, and potassium sulfate, thereby reducing the side effects that may be caused therefrom (e.g., body fluid and electrolyte imbalance). The reduction of the contents thereof can lead to reducing the actually administered amount of tablet(s), thereby being capable of improving patients' compliance. In addition, the composition of the present invention exhibits similar or improved bowel-cleansing effects compared to conventional formulations, along with reducing the occurrence of side effects and improving the patients' compliance.

In the bowel-cleansing composition according to the present invention, the anhydrous magnesium sulfate may be present in a unit dose of 1000˜1300 mg, preferably 1000˜1225 mg. The potassium sulfate may be present in a unit dose of 2000˜2500 mg, preferably 2100˜2400 mg. The anhydrous sodium sulfate may be present in a unit dose of 11500˜13500 mg, preferably 11500˜13400 mg.

In the bowel-cleansing composition according to the present invention, the prucalopride may be present in an amount of 0.01˜0.02% by weight, preferably 0.01˜0.015% by weight, based on the total weight of anhydrous magnesium sulfate, potassium sulfate, and anhydrous sodium sulfate. The sodium picosulfate may be present in an amount of 0.05˜0.15% by weight, preferably 0.05˜0.1% by weight, based on the total weight of anhydrous magnesium sulfate, potassium sulfate, and anhydrous sodium sulfate.

The bowel-cleansing composition according to the present invention may further comprise simethicone. Simethicone works as an adjunct to the bowel-cleansing composition of the present invention with the purpose of diminishing foam formation and improving visualization during colonoscopy.

The bowel-cleansing composition according to the present invention may be in a solid formulation for oral administration, but not limited thereto. For example, the bowel-cleansing composition according to the present invention, may be in the form of one or more tablets. The tablets may be prepared by mixing said components and then compressing into tablets. If necessary, the resulting tablets may be coated with an appropriate coating material.

In an embodiment, the bowel-cleansing composition according to the present invention does not comprise polyethylene glycol (PEG). That is, the bowel-cleansing composition of the present invention does not comprise polyethylene glycol (PEG), thereby avoiding the PEG-derived drawbacks, such as large amounts of dose (e.g., 2 to 4 liters or more), burdens due to unpleasant taste, and so on.

Hereinafter, the present invention will be described in more detail through Examples and Experimental Examples. However, these Examples and Experimental Examples are provided for illustration purposes only and are not intended to limit the scope of the invention.

### EXAMPLES: PREPARATION OF BOWEL-CLEANSING COMPOSITIONS

The sulfate components (anhydrous magnesium sulfate, potassium sulfate, and anhydrous sodium sulfate) along with prucalopride or sodium picosulfate, in the amounts according to the following table 1, were completely dissolved in 425 mL of purified water by stirring at room temperature at 400 rpm for 30 minutes. Comparative Example 1 is purified water, which was used as a vehicle. Comparative Example 2 is the commercially approved product, which contains a combination of sodium picosulfate, magnesium oxide, and citric acid in the amount of unit dose. Comparative Example 3 is Orafang® tablet (Insurance code 659901460) in the amount of unit dose. Example 1 is a combination of 85% by weight of the sulfate components in the unit dose of Orafang® tablet (Comparative Example 2) and prucalopride. Example 2 is a combination of 85% by weight of the sulfate components in the unit dose of Orafang® tablet (Comparative Example 2) and sodium picosulfate. Example 3 is a combination of 75% by weight of the sulfate components in the unit dose of Orafang® tablet (Comparative Example 2) and sodium picosulfate.

TABLE 1

| | Comparative Example | | | Example | | |
|---|---|---|---|---|---|---|
| | 1 | 2 | 3 | 1 | 2 | 3 |
| Aanhydrous | — | — | 1440.0 | 1224.0 | 1224.0 | 1080.0 |
| magnesium | | | | | | |
| sulfate (mg) | | | | | | |
| Potassium | — | — | 2815.0 | 2392.7 | 2392.7 | 2111.3 |
| sulfate (mg) | | | | | | |
| Anhydrous sodium | — | — | 15750 | 13387.5 | 13387.5 | 11775.0 |
| sulfate (mg) | | | | | | |
| Simethicone (mg) | — | — | 160.0 | 160.0 | 160.0 | 160.0 |
| Sodium | — | 10.0 | — | — | 10.0 | 10.0 |
| picosulfate (mg) | | | | | | |
| Prucalopride (mg) | — | — | — | 2 | — | — |
| Pagnesium | — | 3500 | — | — | — | — |
| oxide (mg) | | | | | | |
| Citric acid (mg) | — | 12000 | — | — | — | — |

### Experimental Example 1: Measurement of Osmolality

Korean Pharmacopoeia Dissolution Test Solution 1 (a solution of sodium chloride (2.0 g) and hydrochloric acid (7.0 mL) in water (1000 mL), approximately pH 1.2) and Korean Pharmacopoeia Dissolution Test Solution 2 (a mixed solution of phosphate buffer and water in the weight ratio of 1:1, pH 6.8) were used as a simulated gastric fluid (SGF) and a simulated intestinal fluid (SIF), respectively. Each test solution was added under stirring to the SGF (50 mL) and each sample was taken therefrom. And then, the SIF (100 mL) was added thereto and each sample was taken therefrom. And then, purified water (250 mL) was added thereto and each sample was taken therefrom. The addition of purified water and sampling process were repeated four more times. The osmolality of each sample (50 uL) was measured with an osmometer (Gonotec Osmomat® 3000d).

The results are shown in the following table 2. As shown in Table 2 below, the test solutions of Examples 1, 2, and 3 were diluted showing the osmolality between the osmolality of the test solution of Comparative Example 2 and the osmolality of the test solution of Comparative Example 3, according to the dilutions with the SGF, the SIF, and purified water. These results show that, even when the contents of sodium sulfate, magnesium sulfate, and potassium sulfate were reduced, the osmolality sufficient for washing out stools in the intestines can be obtained.

TABLE 2

| | Test | | | | | | | |
|---|---|---|---|---|---|---|---|---|
| | solution | SGF | SIF | Purified water (mL) | | | | |
| mOsm/kg | per se | (50 mL) | (100 mL) | 250 | 250 | 250 | 250 | 250 |
| Example 1 | 550 | 532 | 439 | 371 | 285 | 219 | 184 | 159 |
| Example 2 | 565 | 542 | 448 | 389 | 299 | 225 | 189 | 163 |
| Example 3 | 481 | 477 | 417 | 347 | 225 | 204 | 171 | 147 |
| Comparative | 423 | 338 | 243 | 105 | 68 | 51 | 42 | 37 |
| Example 2 | | | | | | | | |
| Comparative | 644 | 606 | 520 | 451 | 300 | 275 | 201 | 171 |
| Example 3 | | | | | | | | |

### Experimental Example 2: Bowel-Cleansing Effects in Animal Model

14-week-old male SD rats (Samtako Inc.) were kept in a laboratory environment under the conditions of a temperature of 22±3° C., a relative humidity of 50±10%, and a 12-hour light/dark cycle, while allowing to freely consume food and water. After an adaptation period of 7 days, the rats were used in the experiment.

The test materials of each example and comparative example were orally administered to the SD rats (n=3 per group). Considering a divided therapy, the test materials were orally administered in two times. In consideration of the difference in bowel-cleansing time between humans and rats, the administration interval was set at 70% of the divided therapy interval for a conventional bowel-cleaning formulation. In this experiment, the dose to the animal model was 1.5% of the human dose as shown in Table 3 below. After the administration of each test material, additional water was administered twice at 20-minute intervals. Seven hours after the first administration of each test material, the second administration thereof was carried out and then additional water was administered twice at 20-minute intervals. 10 hours after the first administration of each test material, the animals were sacrificed, and the abdomen was opened so as to isolate the colons. The colon cleanliness for the whole colons and the dissection colons and the colon liquid status were observed and photographed. From the photographs, colon cleanliness was evaluated through scoring on a 3-point scale (“good”, 3 points: empty colon or clear liquid colon; “moderate”, 2 points: brown liquid or loose stool; “bad”, 1 point: presence of semi-solid or hard stool). The average value of the whole colon cleansing score multiplied by a weight of 1 and the dissection cleansing colon score by a weight of 2 was used as a colon cleansing score of the group. Data were expressed as mean±standard deviation. Normality was assumed for the experimental results, which were analyzed with parametric multiple comparison procedures. If the parametric one-way ANOVA results were significant, a post-hoc test was performed using Dunnett's multiple comparison test. Statistical analysis was performed using Prism 5 (GraphPad Software Inc., San Diego, CA, USA). When the p value was less than 0.05, it was considered as being statistically significant (*p<0.05, ***p<0.001 vs Comparative Example 1; ###p<0.001 vs Comparative Example 3).

TABLE 3

| | Comparative Example | | | Example | | |
|---|---|---|---|---|---|---|
| | 1 | 2 | 3 | 2 | 1 | 2 |
| Anhydrous | — | — | 21.60 | 18.36 | 18.36 | 16.20 |
| magnesium | | | | | | |
| sulfate (mg) | | | | | | |
| Potassium | — | — | 42.22 | 35.89 | 35.89 | 31.67 |
| sulfate (mg) | | | | | | |
| Anhydrous sodium | — | — | 236.25 | 200.81 | 200.81 | 176.63 |
| sulfate (mg) | | | | | | |
| Simethicone (mg) | — | — | 2.40 | 2.40 | 2.40 | 2.40 |
| Sodium | — | 0.15 | — | — | 0.15 | 0.15 |
| picosulfate (mg) | | | | | | |
| Prucalopride (mg) | — | — | — | 0.03 | — | — |
| Magnesium | — | 52.50 | — | — | — | — |
| oxide (mg) | | | | | | |
| Citric acid (mg) | — | 180.00 | — | — | — | — |

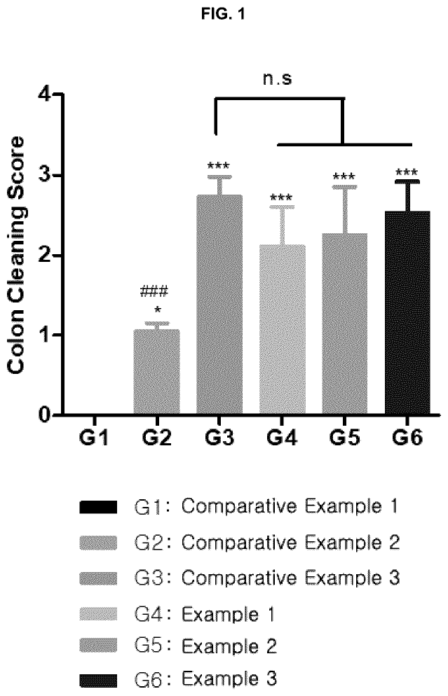

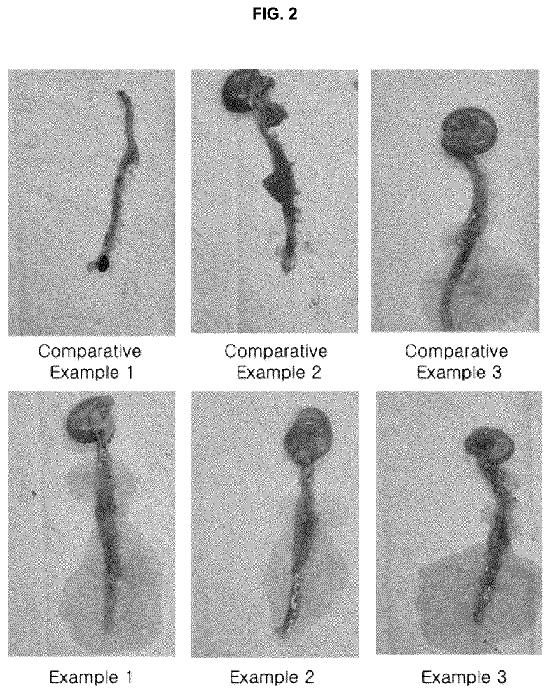

As shown in FIG. 1, the groups administered with the test materials of Examples 1, 2, and 3 showed significantly higher colon cleansing scores compared to the normal control group (Comparative Example 1), while the groups did not show a statistically significant difference from the positive control group (Comparative Example 3). From these results, it can be confirmed that the compositions of the present invention exhibit comparable bowel-cleansing effects. The results obtained by evaluating the bowel-cleansing levels in each group are shown in FIG. 2. From the results thereof, almost residual feces in the colon were not removed in the group administered with the test material of Comparative Example 2; and some residual feces remained in the group administered with the test material of Comparative Example 3. In contrast, stools in the colons were effectively removed in the groups administered with the test materials of Examples 1 to 3. These results show that the improvement of compliance according to the present invention also leads to maintaining or improving the bowel cleansing effects.

Therefore, the composition of the present invention can reduce the contents of sodium sulfate, magnesium sulfate, and potassium sulfate, thereby reducing the side effects that may be caused therefrom (e.g., body fluid and electrolyte imbalance). The reduction of the contents thereof can lead to reducing the actually administered amount of tablet(s), thereby being capable of improving patients' compliance. In addition, the composition of the present invention exhibits similar or improved bowel-cleansing effects compared to conventional formulations, along with reducing the occurrence of side effects and improving the patients' compliance. Therefore, the composition of the present invention can reduce the burdens on the users.

1. A bowel-cleansing composition comprising anhydrous magnesium sulfate, potassium sulfate, and anhydrous sodium sulfate, in combination with prucalopride or sodium picosulfate.

2. The bowel-cleansing composition as claimed in claim 1, wherein the anhydrous magnesium sulfate is present in a unit dose of 1000 to 1300 mg.

3. The bowel-cleansing composition as claimed in claim 1, wherein the potassium sulfate is present in a unit dose of 2000 to 2500 mg.

4. The bowel-cleansing composition as claimed in claim 1, wherein the anhydrous sodium sulfate is present in a unit dose of 11500 to 13500 mg.

5. The bowel-cleansing composition as claimed in claim 1, wherein the prucalopride is present in an amount of 0.01 to 0.02% by weight, based on the total weight of anhydrous magnesium sulfate, potassium sulfate, and anhydrous sodium sulfate.

6. The bowel-cleansing composition as claimed in claim 1, wherein the sodium picosulfate is present in an amount of 0.05 to 0.15% by weight, based on the total weight of anhydrous magnesium sulfate, potassium sulfate, and anhydrous sodium sulfate.

7. The bowel-cleansing composition as claimed in claim 1, further comprising simethicone.

8. The bowel-cleansing composition as claimed in claim 1, which is in a solid formulation for oral administration.

9. The bowel-cleansing composition as claimed in claim 1, wherein the solid formulation for oral administration is in the form of one or more tablets.

10. The bowel-cleansing composition as claimed in claim 1, wherein the composition does not comprise polyethylene glycol.
